(19) United States
(12) Patent Application Publication
(10) Pub. No.: US 2005/0215876 A1
(43) Pub. Date: Sep. 29, 2005

(54) Method and system for automatic image adjustment for in vivo image diagnosis

(75) Inventors: Shoupu Chen, Rochester, NY (US); Nathan D. Cahill, West Henrietta, NY (US); Lawrence A. Ray, Rochester, NY (US)

Correspondence Address: Pamela R. Crocker;Patent Legal Staff, Eastman Kodak Company, 343 State Street, Rochester, NY, 14650-2201, US

(73) Assignee: Eastman Kodak Company

(21) Appl. No.: 10/809,004

(22) Filed: Mar. 25, 2004

(51) Int. Cl. A61B 5/05

(52) U.S. Cl. 600/407

(57) ABSTRACT

A digital image processing method for exposure adjustment of in vivo images that includes the steps of acquiring in vivo images; detecting any crease feature found in the in vivo images; preserving the detected crease feature; and adjusting exposure of the in vivo images with the detected crease feature preserved.

### FIELD OF THE INVENTION

The present invention relates generally to an endoscopic imaging system and, in particular, to image exposure adjustment of in vivo images.

### BACKGROUND OF THE INVENTION

Several in vivo measurement systems are known in the art. They include swallowed electronic capsules which collect data and which transmit the data to an external receiver system. These capsules, which are moved through the digestive system by the action of peristalsis, are used to measure pH (“Heidelberg” capsules), temperature (“CoreTemp” capsules) and pressure throughout the gastro-intestinal (GI) tract. They have also been used to measure gastric residence time, which is the time it takes for food to pass through the stomach and intestines. These capsules typically include a measuring system and a transmission system, wherein the measured data is transmitted at radio frequencies to a receiver system.

U.S. Pat. No. 5,604,531, assigned to the State of Israel, Ministry of Defense, Armament Development Authority, and incorporated herein by reference, teaches an in vivo measurement system, in particular an in vivo camera system, which is carried by a swallowed capsule. In addition to the camera system there is an optical system for imaging an area of the GI tract onto the imager and a transmitter for transmitting the video output of the camera system. The capsule is equipped with a number of LEDs (light emitting diodes) as the lighting source for the imaging system. The overall system, including a capsule that can pass through the entire digestive tract, operates as an autonomous video endoscope. It images even the difficult to reach areas of the small intestine.

U.S. patent application No. 2003/0023150 A1, assigned to Olympus Optical Co., LTD., and incorporated herein by reference, teaches a design of a swallowed capsule-type medical device which is advanced through the inside of the somatic cavities and lumens of human beings or animals for conducting examination, therapy, or treatment. Signals including images captured by the capsule-type medical device are transmitted to an external receiver and recorded on a recording unit. The images recorded are retrieved in a retrieving unit, displayed on the liquid crystal monitor and to be compared by an endoscopic examination crew with past endoscopic disease images that are stored in a disease image database.

One problem associated with the capsule imaging system is a non-uniform lighting over the imaging area due to the nature of this miniature device. Especially, when the capsule travels along a tube-like anatomical structure, the field of view of the camera system covers a section of the anatomical structure inner wall which is nearly parallel with the camera optical axis. Obviously, in this field of view, part of the anatomical structure inner wall away from the capsule receives less photon flux than that of the anatomical structure inner wall close to the capsule. The resultant is a non-uniform photon flux field. In return, part of the image produced by the camera image sensor is either under exposure or over exposure depends on how the camera is calibrated. Therefore, details of texture and color will be lost, which not only affects physicians' ability of abnormality diagnosis using these in vivo images, but also reduces the effectiveness of neighboring in vivo image stitching in applications such image mosiacing.

In general, in order to maximize the use of photon flux, the in vivo camera is calibrated such that there will be no over exposure in the captured images. Thus the non-uniform photon flux distribution results in under exposure in various areas of certain in vivo images. This under exposure of in vivo image is similar to the light falloff in regular photographic images.

U.S. patent application No. 2003/0007707 A1, assigned to Eastman Kodak Company, and incorporated herein by reference, teaches a method for compensating for light falloff caused by the non-uniform exposure which is produced by lenses at their focal plane when imaging a uniformly lit surface. For instance, the light from a uniformly gray wall perpendicular to the camera optical axis will pass through a lens and form an image that is brightest at the center and dims radially. When the lens is an ideal thin lens, the intensity of light in the image will form an intensity pattern described by cos⁴ of the angle between the optical axis of the lens and the point in the image plane. The visible effect of this phenomenon is referred to as falloff. The light compensating method taught in 0007707 describes a compensation function that relies on the value of the distance from a pixel location to the center of the image. Such a method is particularly useful for falloffs caused by lenses distortions. Invention 0007707 teaches a compensation equation:

$$fcm ( x , y ) = \frac{4 * cvs}{log 2} log ( cos ( {tan}^{- 1} ( \frac{dd}{f} ) ) ) .$$

Where dd is the distance in pixels from the (x,y) position to the center of the digital image and cvs is the number of code value per stop of exposure (cvs indicates scaling of the log exposure metric). The parameter f represents the focal length of a lens (in pixels) for which the falloff compensator will correct the falloff. This method is however less desirable for problems caused by non-uniform photon flux field when the endoscopic capsule traveling alone the GI tract, because regions with inadequate exposure do not have the geometric properties stated in the aforementioned equation.

Also the principal advantage of the invention described in 0007707 is that a falloff compensation may be applied to a digital image in such a manner that the balance of the compensated digital image is similar to that of the original digital image, which results in a much more pleasing effect that sometimes may causing problems such as blurring boundaries.

There is a need therefore for an improved endoscopic imaging system that overcomes the problems set forth above.

These and other aspects, objects, features and advantages of the present invention will be more clearly understood and appreciated from a review of the following detailed description of the preferred embodiments and appended claims, and by reference to the accompanying drawings.

### SUMMARY OF THE INVENTION

The need is met according to the present invention by providing a digital image processing method for exposure adjustment of in vivo images that includes the steps of acquiring in vivo images; detecting any crease feature found in the in vivo images; preserving the detected crease feature; and adjusting exposure of the in vivo images with the detected crease feature preserved.

### BRIEF DESCRIPTION OF THE DRAWINGS

FIG. 1 (PRIOR ART) is a block diagram illustration of an in vivo camera system.

FIG. 2A is an illustration of the concept of an examination bundle of the present invention.

FIG. 2B is an illustration of the concept of an examination bundlette of the present invention.

FIG. 3A is a flowchart illustrating information flow of the real-time abnormality detection method in the copending application.

FIG. 3B is a flowchart illustrating information flow of the in vivo image adjustment for diagnosis of the present invention.

FIG. 4 is a schematic diagram of an examination bundlette processing hardware system useful in practicing the present invention.

FIG. 5 is a flowchart illustrating the in vivo image adjustment method of the present invention.

FIG. 6 is a flowchart illustrating the exposure correction and cross boundary smoothing method of the present invention.

FIG. 7A is a schematic diagram of a binary image.

FIG. 7B is a schematic diagram of a mask image.

FIG. 7C is a schematic diagram of a skeleton image.

FIG. 7D is a schematic diagram of a binary image.

FIG. 8 is a collection of patterns.

FIG. 9A is a schematic diagram of an intermediate mask image.

FIG. 9B is a schematic diagram of a mask image.

FIG. 10A is a schematic diagram of a smoothing band image.

FIG. 10B is a schematic diagram of a one dimensional line in the smoothing band.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

During a typical examination of a body lumen, the in vivo camera system captures a large number of images. The images can be analyzed individually, or sequentially, as frames of a video sequence. An individual image or frame without context has limited value. Some contextual information is frequently available prior to or during the image collection process; other contextual information can be gathered or generated as the images are processed after data collection. Any contextual information will be referred to as metadata. Metadata is analogous to the image header data that accompanies many digital image files.

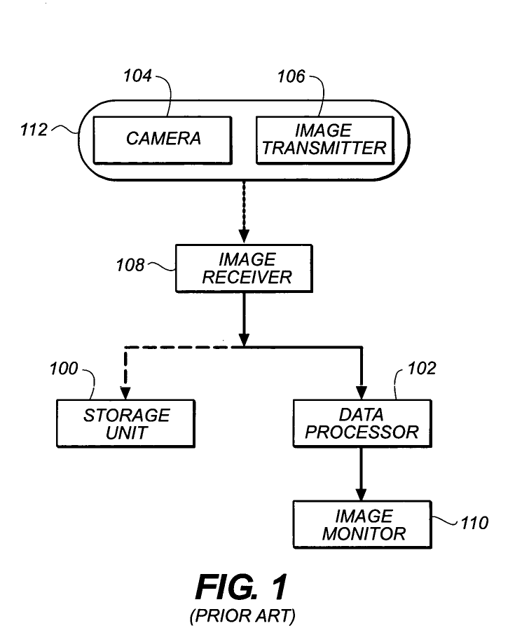

FIG. 1 shows a block diagram of the in vivo video camera system described in U.S. Pat. No. 5,604,531. The system captures and transmits images of the GI tract while passing through the gastro-intestinal lumen. The system contains a storage unit 100, a data processor 102, a camera 104, an image transmitter 106, an image receiver 108, which usually includes an antenna array, and an image monitor 110. Storage unit 100, data processor 102, image monitor 110, and image receiver 108 are located outside the patient's body. Camera 104, as it transits the GI tract, is in communication with image transmitter 106 located in capsule 112 and image receiver 108 located outside the body. Data processor 102 transfers frame data to and from storage unit 100 while the former analyzes the data. Processor 102 also transmits the analyzed data to image monitor 110 where a physician views it. The data can be viewed in real time or at some later date.

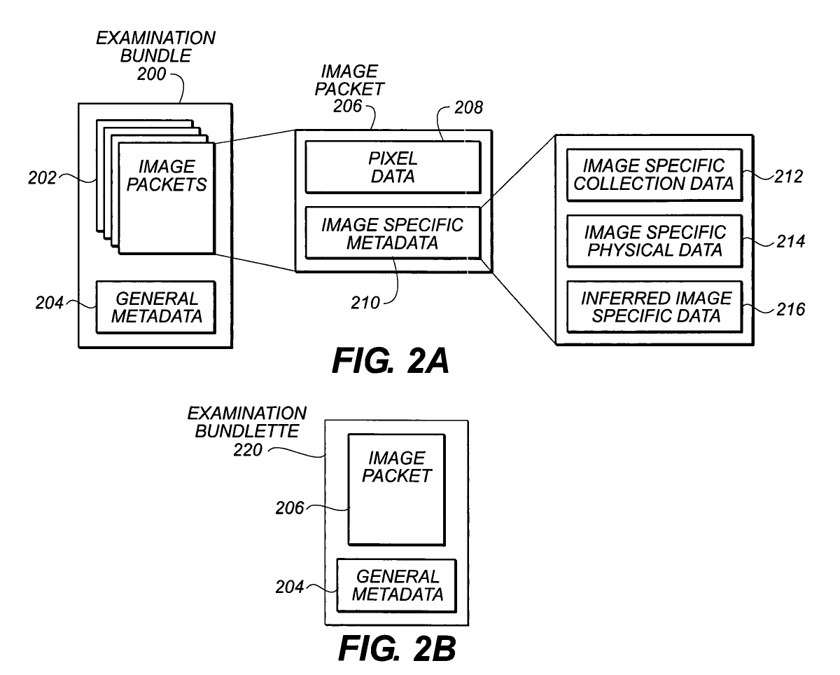

Referring to FIG. 2A, the complete set of all images captured during the examination, along with any corresponding metadata, will be referred to as an examination bundle 200. The examination bundle 200 consists of a collection of image packets 202 and a section containing general metadata 204.

An image packet 206 comprises two sections: the pixel data 208 of an image that has been captured by the in vivo camera system, and image specific metadata 210. The image specific metadata 210 can be further refined into image specific collection data 212, image specific physical data 214 and inferred image specific data 216. Image specific collection data 212 contains information such as the frame index number, frame capture rate, frame capture time, and frame exposure level. Image specific physical data 214 contains information such as the relative position of the capsule when the image was captured, the distance traveled from the position of initial image capture, the instantaneous velocity of the capsule, capsule orientation, and non-image sensed characteristics such as pH, pressure, temperature, and impedance. Inferred image specific data 216 includes location and description of detected abnormalities within the image, and any pathologies that have been identified. This data can be obtained either from a physician or by automated methods.

The general metadata 204 contains such information as the date of the examination, the patient identification, the name or identification of the referring physician, the purpose of the examination, suspected abnormalities and/or detection, and any information pertinent to the examination bundle 200. It can also include general image information such as image storage format (e.g., TIFF or JPEG), number of lines, and number of pixels per line.

Referring to FIG. 2B, the image packet 206 and the general metadata 204 are combined to form an examination bundlette 220 suitable for real-time abnormality detection.

It will be understood and appreciated that the order and specific contents of the general metadata or image specific metadata may vary without changing the functionality of the examination bundle.

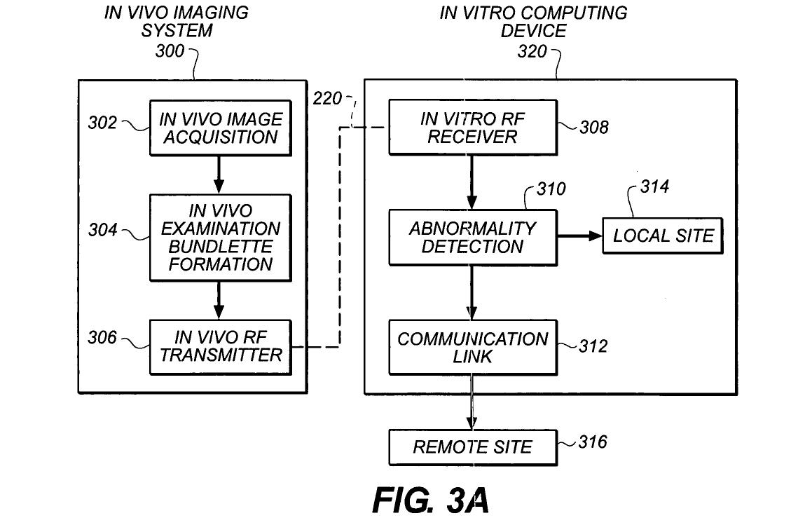

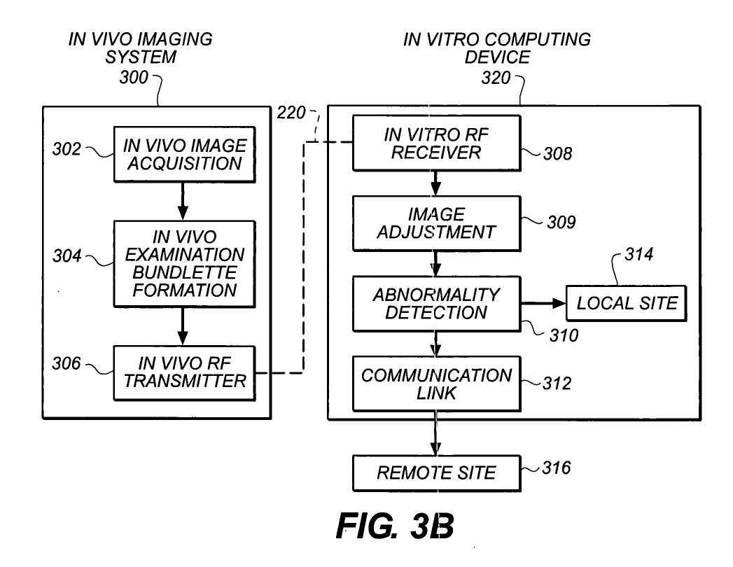

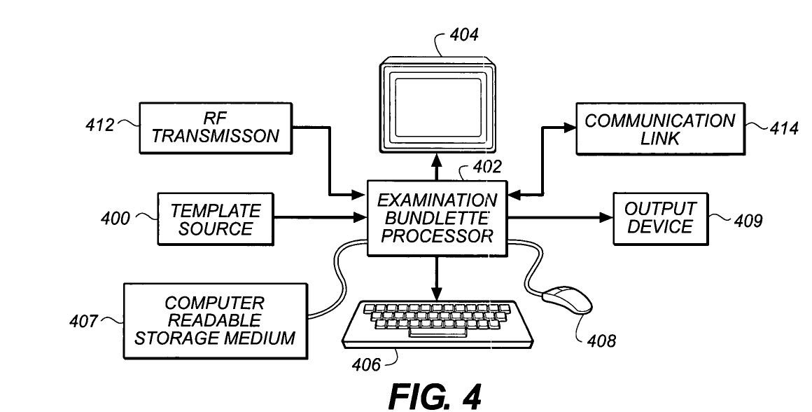

Referring now to FIG. 3A, an exemplary application of the capsule in vivo imaging system is described. FIG. 3 is a flowchart illustrating a real-time automatic abnormality detection method of the present invention. In FIG. 3A, an in vivo imaging system 300 can be realized by using systems such as the swallowed capsule described in U.S. Pat. No. 5,604,531 for the present invention. An in vivo image 208 is captured in an in vivo image acquisition step 302. In a step of In Vivo Examination Bundlette Formation 304, the image 208 is combined with image specific data 210 to form an image packet 206. The image packet 206 is further combined with general metadata 204 and compressed to become an examination bundlette 220. The examination bundlette 220 is transmitted to a proximal in vitro computing device through radio frequency in a step of RF transmission 306. An in vitro computing device 320 is either a portable computer system attached to a belt worn by the patient or in near proximity. Alternatively, it is a system such as shown in FIG. 4 and will be described in detail later. The transmitted examination bundlette 220 is received in the proximal in vitro computing device in a step of In Vivo RF Receiver 308.

Data received in the in vitro computing device is examined for any sign of disease in a step of Abnormality detection 310. Details of the step of abnormality detection can be found in commonly assigned, co-pending U.S. patent application Ser. No. 10/679,711, entitled “Method And System For Real-Time Automatic Abnormality Detection For In Vivo Images” and filed on 6 Oct. 2003 in the names of Shoupu Chen, Lawrence A. Ray, Nathan D. Cahill and Marvin M. Goodgame, and which is incorporated herein by reference.

Note that unlike taking photographic images in natural scenes (indoor or outdoor), in vivo imaging takes place inside the GI tract which is a controlled environment and in general is an open space within the field of the view of the camera. A controlled environment means that there are no sources of lighting other than that from the LEDs of the capsule. An open space implies that there should be no occlusions that cause shadows (under exposure). Also, the reflectance should be the same locally along the GI tract inner wall in general, at least with the same order of magnitude. (This is not the case in real world where the reflectance of photographic objects could vary dramatically causing darker or brighter areas in the resultant images.) Thus, in an ideal case, an in vivo image should not present significant brightness differences in different areas. In reality, because of the uneven photon flux field generated by the limited lighting source, under exposure areas (low brightness areas) exist. Those low brightness areas need to be corrected to become brighter. While in photographic images of natural scenes (indoor or outdoor), low brightness areas could be a result of low reflection of a dark object surface which should not be corrected in an image.

FIG. 3B shows a diagram of information flow of the present invention. To ensure an effective detection and diagnosis of abnormality, images from RF Receiver 308 are exposure adjusted in a step of Image adjusting 309 before the abnormality detection 310 takes place (see FIG. 3B).

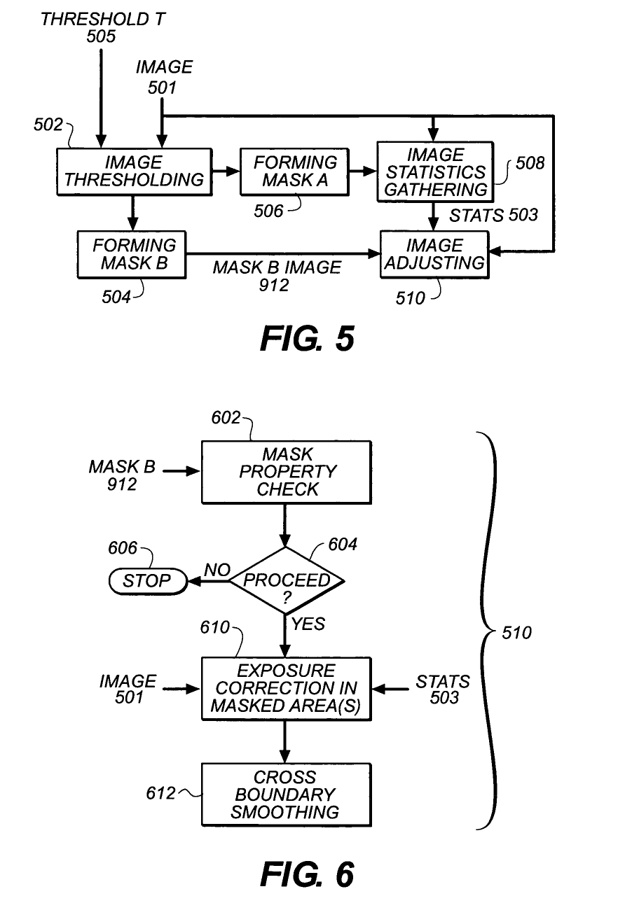

The step of Image adjusting 309 is detailed in FIG. 5. Denote image 501 received from RF receiver 308 by I and its pixel by I(m, n), where m=0, . . . M−1, n=0, . . . N−1, M is the number of rows, and N is the number of columns. To automatically find if an image has under exposure regions, a step of image thresholding 502 is utilized. A threshold T (505) is established through a supervised learning. A supervised learning here means learning in vivo image characteristics by applying statistical analysis to a large number of in vivo images. Statistical analysis includes mean or median intensity analysis, and intensity deviation etc. An exemplary threshold value could be T=mean(I)−K*std(I) where mean(I) returns mean brightness value of the image, std(I) returns the standard deviation value of the image, and K is a coefficient. An exemplary value of K is 3. The output of step 502 is a threshold image I_{B} and its pixel is expressed as I_{B} (m, n). If a pixel value at location (m, n) is less than T (505), then I_{B} (m, n)=1, otherwise, I_{B} (m, n)=0.

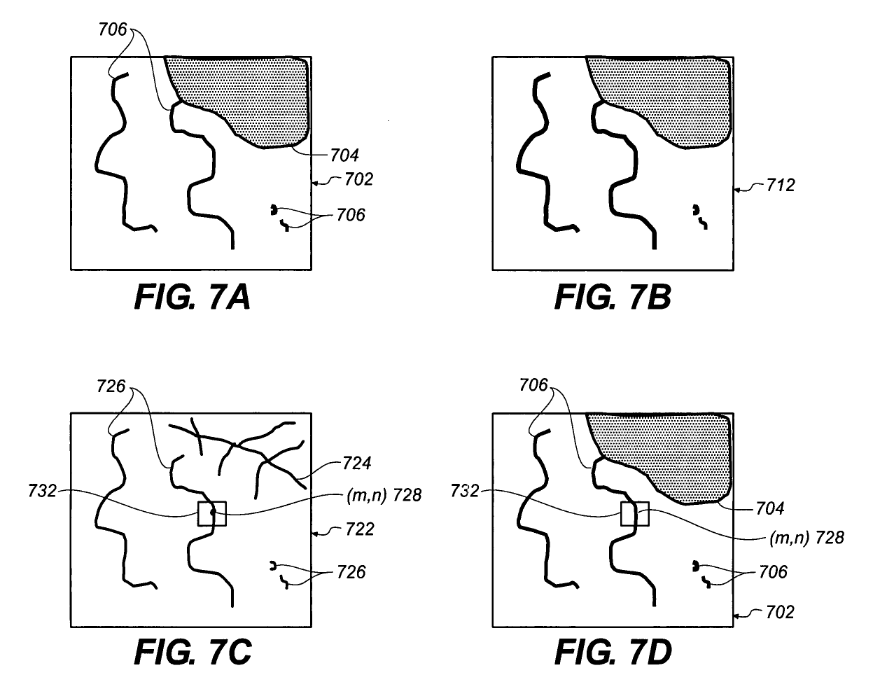

FIG. 7A shows an exemplary threshold image I_{B} (702). The value of pixels I_{B}(m, n) in regions 704, and 706 are one indicating that corresponding pixels, I(m, n), in image I have lower brightness value than T (505). Note that image I_{B} 702 displays exemplary one-valued regions 706 indicating the corresponding low brightness areas in image I (501) caused by crease features where light rays are unable to reach directly in certain anatomical structures of the GI tract. Image I_{B} 702 also displays exemplary one-valued region 704 indicating a low brightness area in image I (501) caused mainly by the non-uniform photon flux field. The low brightness area in image I (501) corresponding to region 704 is subject to image adjustment to lift the brightness level for better diagnosis.

There are variety methods could be used to lift the brightness of an under exposure area in image I (501). A preferred algorithm is described below.

Referring back to FIG. 5, in a step of Forming mask A (506), the threshold image I_{B} (702) undergoes a morphological opening process to close holes and gaps. The resultant image is named as mask A (712) shown in FIG. 7B, and denoted by I_{MA} and its pixel by I_{MA} (m, n). In a step of Image statistics gathering 508, the following equation is used to get statsA (503):
statsA=F(I∩\overline{I}_{MA}) (1)

where I∩\overline{I}_{MA} is a logical AND operation, \overline{I}_{MA} is the logical inverse of I_{MA}, F(●) is a statistical analysis operation, and statsA (503) is a structure containing mean, median and other statistical quantities of the operand which is the result of the logical AND operation, I∩\overline{I}_{MA}. The structure is a C language like data type and statsA (503) is defined as

```
structure stats
{
    mean;
    median;
    minimum;
    maximum;
} statsA
```

where stats is the structure name and statsA.mean is the mean intensity of I∩\overline{I}_{MA}, statsA.median is the median intensity of I∩\overline{I}_{MA}, statsA.minimum is the minimal intensity of I∩\overline{I}_{MA} and statsA.maximum is the maximal intensity of I∩\overline{I}_{MA}.

Note that the logical AND operation, I∩\overline{I}_{MA}, excludes under exposure pixels in the original image I (501) from the statistical analysis operation F(●). The purpose of this exclusion is to learn the statistics only in the normal exposure regions and the learned statistics will be used in a later procedure to lift the brightness level of under exposure regions so that the final image appears coherent.

Since the image adjustment operation is only applied to regions of under exposure (such as 704) caused by the non-uniform photon flux field, a second mask needs to be formed to exclude low brightness regions (such as 706) that belong to crease features. The second mask, mask B, is formed in a step of Forming mask B (504). The step of Forming mask B (504) is further detailed next.

A first operation of forming mask B (504) is a medial axis transformation that is applied to the threshold image I_{B} (702) (see “Algorithm for image processing and computer vision”, by J. R. Parker, Wiley Computer Publishing, John Wiley & Sons, Inc., 1997). A medial axis transformation defines a unique compressed geometrical representation of an object. The medial axis transformation is also referred to as morphological skeletonization. The morphological skeletonization uses erosion and opening as basic operations. The result of the morphological skeletonization is a skeleton image. Denote the skeleton image by I_{S} and its pixel by I_{S} (m, n). Then I_{S} (m, n)=S(I_{B} (m, n)), where S is the medial axis transformation function. I_{S} (m, n) (722), an exemplary result of applying the medial axis transformation to image I_{B} (702), is shown in FIG. 7C. Note that the thick lines 706 in FIG. 7A become one-valued thin lines 726 in FIG. 7C. The one-valued region 704 in FIG. 7A becomes a set of one-valued thin lines 724. Note also that lines 724, and 726 have a width of one pixel. Obviously, every pixel on lines 724, and 726 in image I_{S} must have a corresponding pixel on lines 704 and 706 in image I_{B}. For lines such as 706, their skeleton lines 726 are medial axes of their own. For regions such as 704, in general, they have a set of skeleton lines 724. The skeleton lines are used to detect crease features in the threshold image. The skeleton lines also guide an erasing operation described below.

Denote the second mask, mask B, by I_{MB} and its pixel by I_{MB} (m, n). First, initialize I_{MB} by letting I_{MB} (m, n)=I_{B} (m, n)|∀m,∀n, where ∀m,∀n means all m and all n. Denote an eraser window 732 by W. Exemplary width and height of the eraser window W(732) are 3w, where w is the average width of lines 706. To determine if a one-valued pixel at location (m, n) of the image I_{MB} belongs to crease features such as lines 706, center the eraser window W 732 at the location (m, n) 728 of I_{S} (in operation, the window W is also centered at the location (m, n) 728 of I_{MB}).

In general, there are various types of patterns of the geometry relationship between the window W(732) and the one-valued pixels that belong to crease features such as lines 706. Four exemplary representations of patterns are shown in FIG. 8 assuming window W732 is centered at location (m,n) 728. The process of detecting crease features is to look for these patterns in the threshold image. In a north-south pattern 804, there are zero-valued pixels above and below line 706. In an east-west pattern 802, there are zero-valued pixels left and right to line 706. In a north west-south east pattern 806, there are zero-valued pixels in the upper left and lower right portions of window W (732). In a north east-south west pattern 808, there are zero-valued pixels in the lower left and upper right portions of window W(732).

When pattern 802 occurs, pixel I_{MB} (m, n) and its associated east-west neighboring one-valued pixels are erased. When pattern 804 occurs, pixel I_{MB} (m, n) and its associated north-south neighboring one-valued pixels are erased. When pattern 806 occurs, pixel I_{MB} (m, n) and its associated north west-south east neighboring one-valued pixels are erased. When pattern 808 occurs, pixel I_{MB} (m, n) and its associated north east-south west neighboring one-valued pixels are erased.

The operation of erosion can be described by the following code:

```
for m = 0; m < M; m++
  for n = 0; n < N; n++
    if (I_{S} (m, n) = = 1)
      center W at I_{MB} (m, n)
        if (any one of the patterns (802, 804, 806, 808) occurs)
          erase I_{MB} (m, n) and its associated neighboring pixels;
        end
    end
  end
end
```

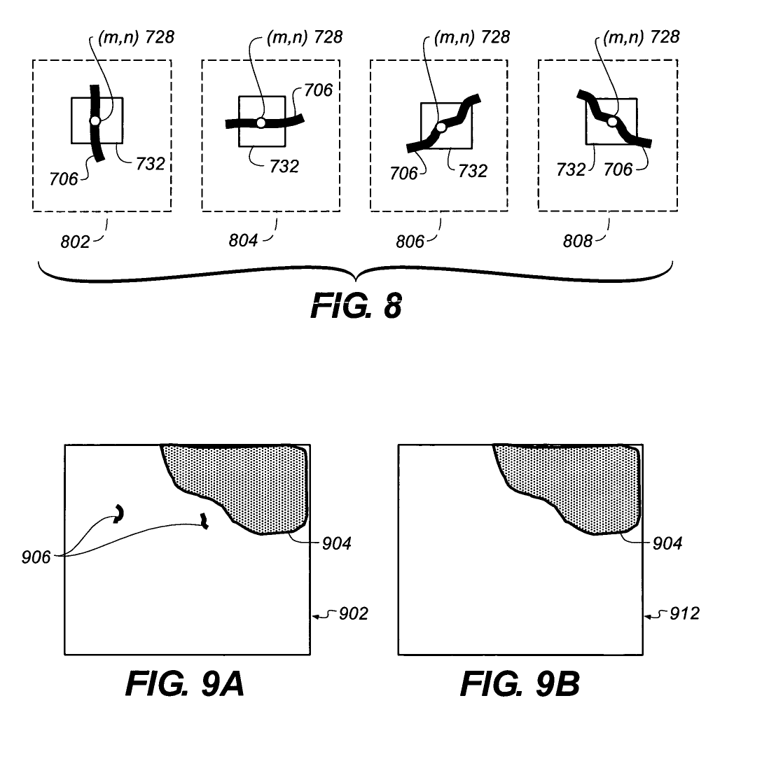

Note that the above erosion operation produces an intermediate mask B image, I_{MB}, 902 shown in FIG. 9A. There may exist residual elements such as tiny regions 906 in FIG. 9A. They can be further eliminated by checking the sizes after clustering the one-valued pixels in I_{MB}.

Those skilled in the art should understand that alternative erasing methods exist. For example, erasing operation can be implemented without performing medial axis transformation by checking more pixels.

Now referring to FIG. 6, there is a flow chart illustrating the steps of image adjustment. One-valued pixels in the mask B image I_{MB} are referred to as foreground pixels. Foreground pixels are grouped to form clusters. A cluster is a non-empty set of one-valued pixels with the property that any pixel within the cluster is also within a predefined distance to another one-valued pixel in the cluster. The present invention groups binary pixels into clusters based upon this definition of a cluster. However, it will be understood that pixels may be clustered on the basis of other criteria.

A cluster may be eliminated if it contains too few one-valued pixels no matter it is a cluster of pixels of crease features or a cluster of pixels of an under exposure region. A cluster contains too few one-valued pixels suggests that the cluster does not have much influence on diagnosis. For example, if the number of pixels in a cluster is less than V, then this cluster is erased from I_{MB}. Example V value could be 10. The above operations are done in a step of Mask property check 602. A query step 604 branches the process to stop 606 if there are no qualified clusters in mask B I_{MB}, or to step 610 if there is at least on qualified cluster. An exemplary qualified mask B I_{MB} 912 is shown in FIG. 9B.

Mask B I_{MB} 912 is now ready to assist applying image adjustment to image I (501) in step 510. Image adjustment is further detailed by steps 610 and 612.

The exposure correction is accomplished in step 610. First, denote an image adjustment process by Φ(●). Denote an adjusted image by I_{adj}. The adjusted image by I_{adj} can be obtained by the following equation:
I_{adj}=(I∩\overline{I}_{MB})∪Φ(I∩I_{MB}) (2)
where \overline{I}_{MB} is the logical inverse of I_{MB}, symbol ∪ is a logic OR operator, and symbol ∩ is a logic AND operator. The operation (I∩I_{MB}) signifies that the adjustment process Φ(●) applies to pixels within region 704 in image I(501). On the other hand, the operation (I∩\overline{I}_{MB}) signifies that the pixels outside the region 704 in image I(501) keep their original value in this stage.

An exemplary of a preferred algorithm of the present invention for the adjustment process Φ(●) is described below:

```
structure stats statsB
statsB = F(I∩I_{MB} )
cf = statsA.median/statsB.median;
for(m = 0; m < M; m++)
{
  for (n = 0; n < N; n++)
  {
    if (I_{MB} (m, n)==1)
    {
      Ĩ_{adj} (m, n) = cfI(m,n);
      if (Ĩ_{adj}(m, n) > statsA.maximum)
        {
          Ĩ_{adj} (m, n) = statsA.maximum;
        }
      }
    }
  }
I_{adj} = (I ∩ \overline{I}_{MB} )∪Ĩ_{adj.}
```

Note that in the above implementation, the adjustment coefficient cf is guaranteed to be greater than or equal to one since statsA=F(I∩\overline{I}_{MA}) and (I∩\overline{I}_{MA}) contains pixels having intensity greater than or equal to T (505), where T=mean(I)−K*std(I). On the other hand, statsB=F(I∩I_{MB}) and (I∩I_{MB}) contains pixels having intensity less than (505).

Notice also that statistics other than median could be used to compute the adjustment coefficient cf. and the adjustment could be applied to individual color channels, (R, G and B), independently. The adjustment operation, Ĩ_{adj}(m, n)=cfI(m, n), in this embodiment is a linear function. But other types of nonlinear functions such as log adjustment or LUT (look up table) also can be used.

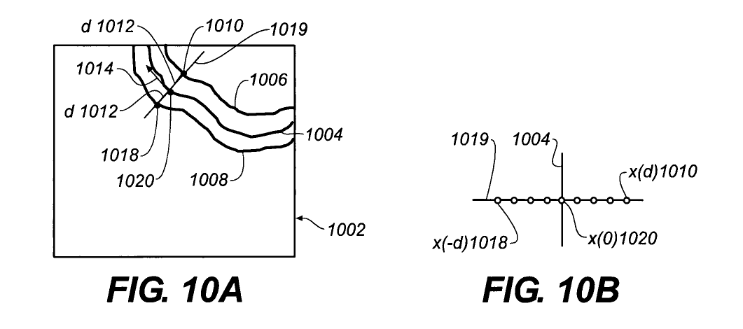

Since the exposure correction is conducted only in areas such as 504 in image I (501), intensity discontinuity between the exposure corrected (adjustment) and uncorrected (non-adjustment) areas may exist along the boundary line such 1004 in FIG. 10A. Line 1004 separates region 904 (same as 504) from the rest of the image. To smooth out intensity discontinuity, a step of Cross boundary smoothing 612 follows the step of Exposure correction in masked area(s) 610.

In FIG. 10A, two lines, two non-intersecting lines 1006 and 1008 define an intensity smoothing band. Lines 1006 and 1008 are on either side of a boundary line 1004 in relation to adjustment and non-adjustment areas for the in vivo image. Lines 1006 and 1008 are formed with respect to line 1004 with a certain distance at each point to form the band width. An exemplary distance is a constant distant d (1012). An exemplary process of forming lines 1006 and 1008 is illustrated as follows. Select a point 1020 on line 1004. Find the tangent arrow 1014 of line 1004 at point 1020. Find a line 1019 that passes point 1020 and is perpendicular to arrow 1014. Find a point 1010 on line 1019 with a distance d (1012) away from point 1020 at one side of line 1004. Find a point 1018 on line 1019 with a distance d (1012) away from point 1020 at the other side of line 1004. Repeating this process for all other points on line 1004 forms two lines 1006 and 1008.

The cross boundary smoothing operation can be realized in one-dimensional space or two-dimensional space. FIG. 10B displays a one-dimensional realization. Denote point 1020 on line 1019 by x(0), point 1018 by x(−d), and point 1010 by x(d). Other points on line 1019 will be named accordingly in the following code of implementation.

$$for ( i = 0 ; i <= d ; i ++ )$$

$$\{ \\ x ( i ) = \frac{1}{2 D + 1} {\sum }_{- D}^{D} x ( i + j ) ; \\ \}$$

$$for ( i = - 1 ; i >= - d ; i -- )$$

$$\{ \\ x ( i ) = \frac{1}{2 D + 1} {\sum }_{- D}^{D} x ( i + j ) ; \\ \}$$

D is less than or equal to d. Exemplary value for D is 1, and 10 for d.

From the above code, it can be seen that the new x(0) is the moving average of pixels from both sides of the boundary line 1014. The influence of pixels from one side to the other side is propagated through newly updated x(i). Starting the process from x(0) helps the propagation of information across the boundary.

The operation described by the above discussion is assumed to be operated in an sRGB space (see Stokes, Anderson, Chandrasekar and Motta, “A Standard Default Color Space for the Internet—sRGB”, http://www.color.org/sRGB.html).

Images in sRGB have already been optimally rendered for video display, typically by applying a 3×3 color transformation matrix and then a gamma compensation lookup table. Any adjustment to the brightness, contrast, and gamma characteristics of an sRGB image will degrade the optimal rendering. If a digital image contained pixel values representative of a linear or logarithmic space with respect to the original scene exposures, the pixel values could be adjusted without degrading any subsequent rendering steps. For those skilled in the art, the ideas and algorithms of the present invention can be applied to spaces such as de-rendered logarithmic space.

FIG. 4 shows an exemplary of an examination bundlette processing hardware system useful in practicing the present invention including a template source 400 and an RF receiver 412 (also 308). The template from the template source 400 is provided to an examination bundlette processor 402, such as a personal computer, or work station such as a Sun Sparc workstation, or a handheld device (e.g., personal digital assistant—PDA). The RF receiver passes the examination bundlette to the examination bundlette processor 402. The examination bundlette processor 402 preferably is connected to a CRT display 404 (which may be a touch-screen display), an operator interface such as a keyboard 406 and a mouse 408. Examination bundlette processor 402 is also connected to computer readable storage medium 407. The examination bundlette processor 402 transmits processed and adjusted digital images and metadata to an output device 409. Output device 409 can comprise a hard copy printer, a long-term image storage device, and a connection to another processor. The examination bundlette processor 402 is also linked to a communication link 414 (also 312) or a telecommunication device connected, for example, to a broadband network.

It is well understood that the transmission of data over wireless links is more prone to requiring the retransmission of data packets than wired links. There is a myriad of reasons for this, a primary one in this situation is that the patient moves to a point in the environment where electromagnetic interference occurs. Consequently, it is preferable that all data from the Examination Bundle be transmitted to a local computer with a wired connection. This has additional benefits, such as the processing requirements for image analysis are easily met, and the primary role of the data collection device on the patient's belt is not burdened with image analysis. It is reasonable to consider the system to operate as a standard local area network (LAN). The device on the patient's belt 100 is one node on the LAN. The transmission from the device on the patient's belt 100 is initially transmitted to a local node on the LAN enabled to communicate with the portable patient device 100 and a wired communication network. The wireless communication protocol IEEE-802.11, or one of its successors, is implemented for this application. This is the standard wireless communications protocol and is the preferred one here. It is clear that the Examination Bundle is stored locally within the data collection device on the patient's belt, as well at a device in wireless contact with the device on the patient's belt. However, while this is preferred, it will be appreciated that this is not a requirement for the present invention, only a preferred operating situation. The second node on the LAN has fewer limitations than the first node, as it has a virtually unlimited source of power, and weight and physical dimensions are not as restrictive as on the first node. Consequently, it is preferable for the image analysis to be conducted on the second node of the LAN. Another advantage of the second node is that it provides a “back-up” of the image data in case some malfunction occurs during the examination. When this node detects a condition that requires the attention of trained personnel, then this node system transmits to a remote site where trained personnel are present, a description of the condition identified, the patient identification, identifiers for images in the Examination Bundle, and a sequence of pertinent Examination Bundlettes. The trained personnel can request additional images to be transmitted, or for the image stream to be aborted if the alarm is declared a false alarm. Details of requesting and obtaining additional images for further diagnosis can be found in commonly assigned, co-pending U.S. patent application Ser. No. (our docket 86570SHS), entitled “Method And System For Real-Time Remote Diagnosis Of In Vivo Images” and filed on 1 Mar. 2004 in the names of Shoupu Chen, Lawrence A. Ray, Nathan D. Cahill, and Marvin M. Goodgame, and which is incorporated herein by reference. To ensure diagnosis accuracy, images to be transmitted are those exposure adjusted in step 309.

The invention has been described in detail with particular reference to certain preferred embodiments thereof, but it will be understood that variations and modifications can be effected within the spirit and scope of the invention.

### PARTS LIST

100 Storage Unit
102 Data Processor
104 Camera
106 Image Transmitter
108 Image Receiver
110 Image Monitor
112 Capsule
200 Examination Bundle
202 Image Packets
204 General Metadata
206 Image Packet
208 Pixel Data
210 Image Specific Metadata
212 Image Specific Collection Data
214 Image Specific Physical Data
216 Inferred Image Specific Data
220 Examination Bundlette
300 In Vivo Imaging system
302 In Vivo Image Acquisition
304 Forming Examination Bundlette
306 RF Transmission
308 RF Receiver
309 Image adjustment
310 Abnormality Detection
312 Communication Connection
314 Local Site
316 Remote Site
320 In Vitro Computing Device
400 Template source
402 Examination Bundlette processor
404 Image display
406 Data and command entry device
407 Computer readable storage medium
408 Data and command control device
409 Output device
412 RF transmission
414 Communication link
501 An image
502 Image Thresholding
503 Stats
504 Forming mask B
505 Threshold
506 Forming mask A
508 Image statistics gathering
510 Image adjusting
602 Mask property check
604 A query
606 Stop
610 Exposure correction in masked area(s)
612 Cross boundary smoothing
702 Binary image
704 A region
706 Lines
712 Mask A
722 Skeleton image
724 Lines
726 Lines
728 A point
732 A window
802 A pattern
804 A pattern
806 A pattern
808 A pattern
816 A dark area
822 A generalized R image
902 An intermediate mask B
904 A region
906 Residuals
912 Mask B image
1002 A smoothing band graph
1004 A line
1006 A line
1008 A line
1010 A point
1012 A distance d
1014 An arrow
1018 A point
1019 A line
1020 A point

1. A digital image processing method for exposure adjustment of in vivo images, comprising the steps of:
a) acquiring in vivo images;
b) detecting any crease feature found in the in vivo images;
c) preserving the detected crease feature; and
d) adjusting exposure of the in vivo images with the detected crease feature preserved.

2. The digital image processing method claimed in claim 1, wherein the step of adjusting exposure of the in vivo images includes the steps of:
d1) thresholding the in vivo images to form a threshold image;
d2) forming a first mask, A, from the threshold image;
d3) forming a second mask, B, from the threshold image;
d4) gathering image statistics with mask A; and
d5) adjusting image exposure with mask B and the gathered statistics of mask A.

3. The digital image processing method claimed in claim 2, wherein the step of adjusting image exposure with mask B and the gathered statistics of mask A further includes the step of forming a smoothing band across an adjustment boundary, and smoothing image pixels in the smoothing band.

4. The digital image processing method claimed in claim 1, wherein detecting the crease feature, further includes the steps of:
b1) forming a skeleton image of the threshold image; and
b2) testing the skeleton image and the threshold image for one or more crease features.

5. The digital image processing method claimed in claim 2, wherein forming a second mask, B, from the threshold image, further includes the steps of:
i.) erasing corresponding pixels of the detected crease feature in the threshold image; and
ii.) erasing any remaining residual elements from the threshold image, wherein the residual elements are tiny regions.

6. The digital image processing method claimed in claim 1, wherein an image area indicated by mask B is intensified using an adjustment coefficient.

7. The digital image processing method claimed in claim 6, wherein the adjustment coefficient is determined by distinct statistics of intensity corresponding to masked areas and unmasked areas of an original image, respectively.

8. The digital image processing method claimed in claim 6, wherein the image area indicated by mask B is intensified using the adjustment coefficient, and said intensification is selected from the group consisting of a linear function, a non-linear function, and a look-up table.

9. The digital image processing method claimed in claim 6, wherein the image area indicated by mask B is monochrome or polychrome.

10. The digital image processing method claimed in claim 3, wherein forming a smoothing band further includes the steps of:
i) forming two non-intersecting lines, one on either side of a boundary line in relation to adjustment and non-adjustment areas for the in vivo image;
ii) defining a width of the smoothing band from the two non-intersecting lines; and
iii) determining intensity of in vivo image pixels on the boundary in the smoothing band from a moving average of in vivo image pixels found on both side of the boundary line;
iv) determining intensity of in vivo image pixels off the boundary in the smoothing band from a moving average of in vivo image pixels newly updated starting from the pixels on the boundary.

11. A digital image processing method for exposure adjustment of in vivo images, comprising the steps of:
a) acquiring the in vivo images using an in vivo video camera system;
b) forming an examination bundlette from the in vivo images acquired with the in vivo video camera system;
c) transmitting the examination bundlette to proximal in vitro computing device(s);
d) processing the examination bundlette; and
e) adjusting exposure of the in vivo images transmitted in the examination bundlette, while simultaneously preserving any crease feature found in the in vivo images.

12. The digital image processing method claimed in claim 11, further comprising the step of notifying a remote site of suspected abnormalities that have been identified in the in vivo images.

13. The digital image processing method claimed in claim 12, wherein a communication channel is provided to the remote site.

14. The digital image processing method claimed in claim 11, wherein the in vivo video camera system comprises a camera having video capture capability; and an optical system for imaging an area of interest onto said camera.

15. The digital image processing method claimed in claim 11, wherein the step of forming an in vivo video camera system examination bundlette includes the steps of:
i.) forming an image packet; and
ii.) forming general metadata.

16. The digital image processing method claimed in claim 11, wherein the in vitro computing device comprises a radio receiver, an examination bundlette processor, and a wireless communication system.

17. The digital image processing method claimed in claim 11, wherein the step of processing the examination bundlette comprises the steps of:
i) decomposing the examination bundlette; and
ii) processing the in vivo images.

18. The digital image processing method claimed in claim 11, wherein the step of adjusting exposure of the in vivo images includes the steps of:
d1) thresholding the in vivo images to form a threshold image;
d2) forming a first mask, A, from the threshold image;
d3) forming a second mask, B, from the threshold image;
d4) gathering image statistics with mask A; and
d5) adjusting image exposure with mask B and the gathered statistics of mask A.

19. The digital image processing method claimed in claim 18, wherein the step of adjusting image exposure with mask B and the gathered statistics of mask A further includes the step of forming a smoothing band across an adjustment boundary, and smoothing image pixels in the smoothing band.

20. The digital image processing method claimed in claim 11, wherein detecting the crease feature, further includes the steps of:
b1) forming a skeleton image of the threshold image; and
b2) testing the skeleton image for one or more crease features.

21. The digital image processing method claimed in claim 18, wherein forming a second mask, B, from the threshold image, further includes the steps of:
i.) erasing corresponding pixels of the detected crease feature in the threshold image; and
ii.) erasing any remaining residual elements from the threshold image, wherein the residual elements are tiny regions.

22. The digital image processing method claimed in claim 11, wherein an image area indicated by mask B is intensified using an adjustment coefficient.

23. The digital image processing method claimed in claim 22, wherein the adjustment coefficient is determined by distinct statistics of intensity corresponding to masked areas and unmasked areas of an original image, respectively.

24. The digital image processing method claimed in claim 22, wherein mask B is intensified using the adjustment coefficient, and said intensification is selected from the group consisting of a linear function, a non-linear function, and a look-up table.

25. The digital image processing method claimed in claim 22, wherein mask B is intensified using the adjustment coefficient is applied to gray-scale or color images.

26. The digital image processing method claimed in claim 19, wherein forming a smoothing band further includes the steps of:
i) forming two non-intersecting lines, one on either side of a boundary line in relation to adjustment and non-adjustment areas for the in vivo image;
ii) defining a width of the smoothing band from the two non-intersecting lines; and
iii) determining intensity of in vivo image pixels on the boundary in the smoothing band from a moving average of in vivo image pixels found on both side of the boundary line;
iv) determining intensity of in vivo image pixels off the boundary in the smoothing band from a moving average of in vivo image pixels newly updated starting from the pixels on the boundary.

27. An examination bundlette processing hardware system for in vivo imaging, comprising:
a) an examination bundlette processor for adjusting exposure of in vivo images while preserving any detected crease feature in the in vivo images;
b) a radio frequency receiver/transmitter connected to the examination bundlette processor for transmitting data packets containing the in vivo images;
c) a communication link connected to the examination bundlette processor for establishing a network link for communication the data packets;
d) a computer readable storage medium connected to the examination bundlette processor for storing the data packets;
e) a display device connected to the examination bundlette processor for providing user interface via a keyboard and/or a mouse, or a touch screen; and
f) an output device connected to the examination bundlette processor for transforming the data packets to another media, wherein the media includes print and storage.

28. The examination bundlette processing hardware system claimed in claim 27, wherein said system is incorporated within a handheld personal digital assistant, (PDA).
